(19) United States
(12) Patent Application Publication
(10) Pub. No.: US 2026/0157996 A1
(43) Pub. Date: Jun. 11, 2026

(54) COMPOSITION FOR MODULATING THE HIPPO-YAP PATHWAY AND PHARMACEUTICAL COMPOSITION FOR PREVENTING OR TREATING CANCER

(71) Applicant: AJOU UNIVERSITY INDUSTRY-ACADEMIC COOPERATION FOUNDATION, Suwon-si (KR)

(72) Inventors: Jung Soon MO, Suwon-si (KR); Han Sol JEONG, Busan (KR); Cho Long KIM, Hwaseong-si (KR)

(73) Assignee: AJOU UNIVERSITY INDUSTRY-ACADEMIC COOPERATION FOUNDATION, Suwon-si (KR)

(21) Appl. No.: 19/395,108

(22) Filed: Nov. 20, 2025

(30) Foreign Application Priority Data
Nov. 27, 2024 (KR) 10-2024-0172008

(51) Int. Cl. A61K 31/365; A61P 35/00

(52) U.S. Cl. CPC A61K 31/365; A61P 35/00

(57) ABSTRACT

The present disclosure relates to a composition for modulating the Hippo-YAP pathway, comprising isoalantolactone as an active ingredient.

### FIELD

The present disclosure relates to a composition for modulating the Hippo-YAP pathway and a pharmaceutical composition for the prevention or treatment of cancer.

### DESCRIPTION OF THE RELATED ART

Cholangiocarcinoma (CCA) is a highly invasive and heterogeneous malignant tumor that originates from the biliary epithelial cells. CCA exhibits an extremely poor prognosis, with a median survival time of approximately six months, due to difficulties in early diagnosis, the aggressive nature of the disease, and its resistance to conventional therapies.

Recent studies have revealed that various signaling pathways, including Notch, Hippo, FGF/FGFR, mTOR, and TGF-β, play pivotal roles in the development of CCA. In particular, Yes-associated protein (YAP), a major effector protein of the Hippo signaling pathway, functions as a transcriptional co-activator that regulates organ size, cell proliferation, and apoptosis, and is known to significantly influence the progression of CCA.

In cholangiocarcinoma tissues, overexpression and nuclear activation of YAP are frequently observed, showing a strong correlation with tumor initiation, progression, and poor prognosis. Accordingly, YAP has attracted considerable attention as a promising therapeutic target for cholangiocarcinoma.

Meanwhile, natural products have long been utilized in the treatment of various diseases in Asian regions, and in recent years, increasing attention has been given to natural compounds with anticancer activity. Isoalantolactone (IALT) is a natural compound extracted from medicinal plants such as I. helenium and Inula racemosa Hook. f, and exhibits diverse pharmacological properties, including anti-inflammatory, antioxidant, antimicrobial, and anticancer activities. In particular, IALT has been reported to inhibit cell proliferation and induce apoptosis in various cancer cell lines, and when used in combination with cisplatin, it effectively overcomes drug resistance in ovarian cancer.

However, to date, research on the effects of IALT on the Hippo-YAP signaling pathway and its molecular mechanisms in cholangiocarcinoma remains insufficient. Therefore, there is a need to develop YAP-targeted therapeutic strategies utilizing IALT to improve the prognosis of cholangiocarcinoma.

The technical background of the present disclosure, Korean Patent Publication No. 10-2024-0086475, relates to novel biomarkers for the diagnosis and prognosis prediction of cholangiocarcinoma and their uses.

### DESCRIPTION

### Technical Problem

The present disclosure is directed to solving the problems of the prior art described above and provides a composition for modulating the Hippo-YAP pathway.

The present disclosure also provides a pharmaceutical composition for the prevention or treatment of cancer.

The present disclosure further provides a kit for the prevention or treatment of cancer, comprising the pharmaceutical composition for the prevention or treatment of cancer.

However, the technical problems to be achieved by the embodiments of the present disclosure are not limited to the technical problems described above, and there may be other technical problems.

### Means for Solving the Problem

As a technical means for achieving the aforementioned technical problems, a first aspect of the present disclosure is directed to providing a composition for modulating the Hippo-YAP pathway, comprising isoalantolactone as an active ingredient.

According to one embodiment of the present disclosure, YAP may be phosphorylated by the isoalantolactone, but is not limited thereto.

According to one embodiment of the present disclosure, cytoplasmic sequestration of YAP may be induced by the isoalantolactone, but is not limited thereto.

According to one embodiment of the present disclosure, the interaction between YAP and TEAD may be inhibited by the isoalantolactone, but is not limited thereto.

According to one embodiment of the present disclosure, the expression of YAP-dependent genes may be inhibited by the isoalantolactone, but is not limited thereto.

According to one embodiment of the present disclosure, the YAP-dependent gene may comprise at least one selected from the group consisting of CYR61 and CTGF, but is not limited thereto.

Furthermore, a second aspect of the present disclosure is directed to providing a pharmaceutical composition for the prevention or treatment of cancer, comprising isoalantolactone as an active ingredient.

According to one embodiment of the present disclosure, apoptosis of cancer cells may be induced by the isoalantolactone, but is not limited thereto.

According to one embodiment of the present disclosure, tumor growth may be inhibited by the isoalantolactone, but is not limited thereto.

According to one embodiment of the present disclosure, the cancer may be selected from the group consisting of cholangiocarcinoma, lung cancer, breast cancer, oral cancer, esophageal cancer, gastric cancer, liver cancer, pancreatic cancer, colorectal cancer, renal cancer, cervical cancer, ovarian cancer, prostate cancer, and combinations thereof, but is not limited thereto.

According to one embodiment of the present disclosure, the cancer may comprise cholangiocarcinoma, but is not limited thereto.

According to one embodiment of the present disclosure, the pharmaceutical composition for the prevention or treatment of cancer may further comprise a pharmaceutically acceptable carrier, but is not limited thereto.

According to one embodiment of the present disclosure, the carrier may comprise one selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, menthol, mineral oil, and combinations thereof, but is not limited thereto.

According to one embodiment of the present disclosure, the pharmaceutical composition for the prevention or treatment of cancer may further comprise an ingredient selected from the group consisting of preservatives, solubilizers, stabilizers, wetting agents, sweeteners, colorants, flavoring agents, salts, buffers, antioxidants, lubricants, emulsifiers, suspending agents, and combinations thereof, but is not limited thereto.

According to one embodiment of the present disclosure, the pharmaceutical composition for the prevention or treatment of cancer may be administered by a method selected from the group consisting of intraperitoneal administration, oral administration, inhalation administration, intravenous administration, intramuscular administration, subcutaneous administration, intradermal administration, intrauterine administration, tumor administration, rectal administration, and combinations thereof, but is not limited thereto.

Furthermore, a third aspect of the present disclosure is directed to providing a kit for the prevention or treatment of cancer, comprising the pharmaceutical composition for the prevention or treatment of cancer according to the second aspect of the present disclosure.

The aforementioned means for solving the problem are merely exemplary and should not be construed as intending to limit the present disclosure. In addition to the exemplary embodiments described above, further embodiments may exist in the drawings and the detailed description of the invention.

### Effect of the Invention

The present invention elucidates a novel mechanism by which isoalantolactone (IALT) modulates the Hippo-YAP pathway, thereby providing its potential as a preventive or therapeutic agent for cancer. Specifically, IALT can effectively inhibit cell growth and proliferation in the cholangiocarcinoma cell line SNU478 by promoting the phosphorylation of YAP and suppressing its nuclear translocation, thereby improving the pathological condition of cancer.

In addition, IALT may act effectively in the prevention or treatment of cancer by inhibiting the expression of YAP-dependent genes, including CYR61 and CTGF. This suggests that IALT can exert a more potent therapeutic effect by suppressing the YAP-TEAD transcriptional complex, which is directly involved in the oncogenic mechanism.

Furthermore, IALT exhibits a significant antitumor effect in vivo by markedly reducing tumor growth in an immunodeficient nude mouse xenograft model. In particular, IALT demonstrates cytotoxic effects on cancer cells through caspase-dependent apoptosis, indicating that it may serve as a selective and effective therapeutic strategy in cancer treatment.

However, the effects that can be obtained from the present disclosure are not limited to the effects described above, and there may be other effects.

### BRIEF DESCRIPTION OF THE DRAWING

FIG. 1A is a diagram showing the chemical structure of isoalantolactone (IALT).

FIG. 1B is a graph showing the inhibitory effect of IALT on cell proliferation in SNU478 cells.

FIG. 1C is a graph showing the distribution of apoptosis in SNU478 cells according to IALT treatment.

FIG. 1D is an immunoblot result showing the changes in the expression of apoptosis-related proteins following IALT treatment.

FIG. 2A is an immunoblot result showing the phosphorylation status of YAP according to the concentration of IALT treatment.

FIG. 2B is an immunofluorescence image showing the intracellular localization of YAP following IALT treatment.

FIG. 2C is an immunoprecipitation result showing the effect of IALT treatment on the interaction between YAP and TEAD proteins.

FIG. 2D is a qRT-PCR result showing the changes in the expression of YAP target genes following IALT treatment.

FIG. 3A is an immunoblot result showing the effect of IALT on YAP phosphorylation in wild-type and LATS1/LATS2-deficient cells.

FIG. 3B is an immunofluorescence image showing the intracellular localization of YAP after IALT treatment in wild-type and LATS1/LATS2-deficient cells.

FIG. 3C is a graph showing the inhibitory effect of IALT on cell viability in LATS1/LATS2-deficient cells.

FIG. 3D is a graph showing the induction of apoptosis by IALT in wild-type and LATS1/LATS2-deficient cells.

FIG. 3E is an immunoblot result showing the changes in the expression of apoptosis-related proteins after IALT treatment in wild-type and LATS1/LATS2-deficient cells.

FIG. 4A shows the inhibitory effect of IALT on colony formation in wild-type and LATS1/LATS2-deficient cells.

FIG. 4B is a wound-healing assay result showing the inhibitory effect of IALT on cell migration in normal and YAP-TEAD-activated cells.

FIG. 4C shows images of excised tumors from nude mice.

FIG. 4D is a graph showing the change in tumor weight following IALT treatment.

FIG. 4E is a graph showing the change in body weight of mice during the IALT treatment period.

FIG. 4F is a graph showing the change in tumor volume following IALT treatment.

FIG. 4G is an immunoblot result showing the changes in YAP phosphorylation and target gene expression in tumor tissues after IALT treatment.

### DETAILED DESCRIPTION

Hereinafter, examples of the present disclosure will be described in detail so as to be easily implemented by those skilled in the art, with reference to the accompanying drawings. However, the present disclosure may be embodied in many different forms and are not limited to the examples to be described herein. In addition, parts not related with the description have been omitted in order to clearly describe the present disclosure in the drawings and throughout the present specification, like reference numerals designate like elements.

Further, throughout this specification, when a certain part is “connected” with the other part, it is meant that the certain part may be “directly connected” with the other part and “electrically connected” with the other part with another element interposed therebetween.

Throughout the present specification, it will be understood that when a certain member is located “on”, “above”, “at the top of”, “under”, “below”, and “at the bottom of” the other member, a certain member is in contact with the other member and another member may also be present between the two members.

Throughout the specification, a case where a part “includes” an element will be understood to imply the inclusion of stated elements but not the exclusion of any other elements unless explicitly described to the contrary.

The terms “about”, “substantially”, and the like to be used in the specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present disclosure. Throughout the present specification, the term of “step to” or “step of” does not mean “step for”.

Throughout the present specification, the term “combinations thereof” included in the expression of the Markush form means one or more mixtures or combinations selected from the group consisting of components described in the expression of the Markush form, and means to include at least one selected from the group consisting of the components.

Throughout the present specification, “A and/or B” means “A or B, or A and B”.

Hereinafter, the composition for modulating the Hippo-YAP pathway of the present disclosure will be described in detail with reference to embodiments, examples, and drawings. However, the present disclosure is not limited to these embodiments, examples, and drawings.

As a technical means for achieving the aforementioned technical problems, a first aspect of the present disclosure is directed to providing a composition for modulating the Hippo-YAP pathway, comprising isoalantolactone as an active ingredient.

The Hippo-YAP pathway plays an important role in various physiological processes such as cell proliferation, differentiation, and organ size regulation, and is particularly known as a key signaling pathway involved in cancer development and progression. The composition for modulating the Hippo-YAP pathway according to the present disclosure can effectively regulate the Hippo-YAP pathway through isoalantolactone and may therefore be applied to the prevention or treatment of cancer.

According to one embodiment of the present disclosure, YAP may be phosphorylated by the isoalantolactone, but is not limited thereto.

Phosphorylation of YAP is a key regulatory mechanism of the Hippo pathway. Phosphorylated YAP is inactivated, thereby suppressing the growth and proliferation of cancer cells. The composition for modulating the Hippo-YAP pathway according to the present disclosure can regulate YAP activity by promoting the phosphorylation of the YAP protein through isoalantolactone.

According to one embodiment of the present disclosure, cytoplasmic sequestration of YAP may be induced by the isoalantolactone, but is not limited thereto.

When YAP is sequestered in the cytoplasm, its translocation to the nucleus is inhibited, resulting in the suppression of its function as a transcriptional co-activator, which can reduce the expression of genes essential for cancer cell growth and survival. The composition for modulating the Hippo-YAP pathway according to the present disclosure can control the function of YAP by regulating its intracellular localization through isoalantolactone.

According to one embodiment of the present disclosure, the interaction between YAP and TEAD may be inhibited by the isoalantolactone, but is not limited thereto.

Inhibition of YAP-TEAD complex formation can effectively suppress the transcription of genes required for cancer cell proliferation and survival. The composition for modulating the Hippo-YAP pathway according to the present disclosure can regulate the expression of cancer-related genes by interfering with the binding between YAP and the TEAD transcription factor through isoalantolactone.

According to one embodiment of the present disclosure, the expression of YAP-dependent genes may be inhibited by the isoalantolactone, but is not limited thereto.

Inhibition of the expression of YAP-dependent genes can effectively suppress various tumor progression processes, including cancer cell proliferation, survival, and metastasis. The composition for modulating the Hippo-YAP pathway according to the present disclosure can regulate the expression of downstream target genes by inhibiting the transcriptional activity of YAP through isoalantolactone.

According to one embodiment of the present disclosure, the YAP-dependent gene may comprise at least one selected from the group consisting of CYR61 and CTGF, but is not limited thereto.

CYR61 and CTGF are key factors involved in tumor progression, including cell proliferation, survival, and angiogenesis. Inhibition of their expression can effectively suppress tumor growth and metastasis. The composition for modulating the Hippo-YAP pathway according to the present disclosure can regulate the expression of specific YAP target genes that play crucial roles in cancer development through isoalantolactone.

Furthermore, a second aspect of the present disclosure is directed to providing a pharmaceutical composition for the prevention or treatment of cancer, comprising isoalantolactone as an active ingredient.

With respect to the pharmaceutical composition for the prevention or treatment of cancer according to the second aspect of the present disclosure, detailed descriptions of the overlapping parts with the first aspect of the present disclosure have been omitted. However, even if the descriptions are omitted, the contents described in the first aspect of the present disclosure can be equally applied to the second aspect of the present disclosure.

The present invention elucidates a novel mechanism by which isoalantolactone (IALT) modulates the Hippo-YAP pathway, thereby presenting its potential as a preventive or therapeutic agent for cancer. Specifically, IALT can effectively inhibit cell growth and proliferation in the cholangiocarcinoma cell line SNU478 by promoting the phosphorylation of YAP and suppressing its nuclear translocation, thereby improving the pathological condition of cancer.

In addition, IALT may act effectively in the prevention or treatment of cancer by inhibiting the expression of YAP-dependent genes, including CYR61 and CTGF. This indicates that IALT can provide a more potent therapeutic effect by suppressing the YAP-TEAD transcriptional complex, which is directly involved in oncogenic mechanisms.

Furthermore, IALT exhibits an excellent antitumor effect in vivo by significantly reducing tumor growth in an immunodeficient nude mouse xenograft model. In particular, IALT induces cytotoxic effects on cancer cells through caspase-dependent apoptosis, suggesting that it may serve as a selective and effective therapeutic strategy in cancer treatment.

According to one embodiment of the present disclosure, apoptosis of cancer cells may be induced by the isoalantolactone, but is not limited thereto.

Apoptosis is a programmed cell death process that eliminates abnormal cells, and the induction of apoptosis by isoalantolactone can serve as an effective strategy in cancer therapy. The pharmaceutical composition for the prevention or treatment of cancer according to the present disclosure can selectively eliminate cancer cells through caspase-dependent apoptosis induced by isoalantolactone.

According to one embodiment of the present disclosure, tumor growth may be inhibited by the isoalantolactone, but is not limited thereto.

Inhibition of tumor growth is one of the most important objectives in cancer treatment, and isoalantolactone can suppress tumor growth through various mechanisms. The pharmaceutical composition for the prevention or treatment of cancer according to the present disclosure can effectively inhibit tumor size and progression through isoalantolactone.

According to one embodiment of the present disclosure, the cancer may be selected from the group consisting of cholangiocarcinoma, lung cancer, breast cancer, oral cancer, esophageal cancer, gastric cancer, liver cancer, pancreatic cancer, colorectal cancer, renal cancer, cervical cancer, ovarian cancer, prostate cancer, and combinations thereof, but is not limited thereto.

According to one embodiment of the present disclosure, the cancer may comprise cholangiocarcinoma, but is not limited thereto.

Cholangiocarcinoma is a malignancy with a very poor prognosis due to late diagnosis, aggressive characteristics, and resistance to current therapies. Isoalantolactone can effectively suppress the progression of cholangiocarcinoma by regulating the Hippo-YAP pathway. The pharmaceutical composition for the prevention or treatment of cancer according to the present disclosure may therefore be particularly effective for the prevention or treatment of cholangiocarcinoma through isoalantolactone.

According to one embodiment of the present disclosure, the pharmaceutical composition for the prevention or treatment of cancer may further comprise a pharmaceutically acceptable carrier, but is not limited thereto.

According to one embodiment of the present disclosure, the carrier may comprise one selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, menthol, mineral oil, and combinations thereof, but is not limited thereto.

According to one embodiment of the present disclosure, the pharmaceutical composition for the prevention or treatment of cancer may further comprise an ingredient selected from the group consisting of preservatives, solubilizers, stabilizers, wetting agents, sweeteners, colorants, flavoring agents, salts, buffers, antioxidants, lubricants, emulsifiers, suspending agents, and combinations thereof, but is not limited thereto.

According to one embodiment of the present disclosure, the pharmaceutical composition for the prevention or treatment of cancer may be administered by a method selected from the group consisting of intraperitoneal administration, oral administration, inhalation administration, intravenous administration, intramuscular administration, subcutaneous administration, intradermal administration, intrauterine administration, tumor administration, rectal administration, and combinations thereof, but is not limited thereto.

Furthermore, a third aspect of the present disclosure is directed to providing a kit for the prevention or treatment of cancer, comprising the pharmaceutical composition for the prevention or treatment of cancer according to the second aspect of the present disclosure.

With respect to the kit for the prevention or treatment of cancer according to the third aspect of the present disclosure, detailed descriptions of the overlapping parts with the first and/or second aspects of the present disclosure have been omitted. However, even if the descriptions are omitted, the contents described in the first and/or second aspects of the present disclosure can be equally applied to the third aspect of the present disclosure.

Hereinafter, the present invention will be explained in more detail through Examples, but the following Examples are for the purpose of illustration only and are not intended to limit the scope of the present disclosure.

### Materials and Methods

### Antibodies

LATS1, LATS2, phosphorylated-LATS1 (Thr1079), MST2, MAP4K4, PARP, MOB1, phosphorylated-MOB1 (Thr35), YAP, phosphorylated-YAP (Ser127), and YAP/TAZ were purchased from Cell Signaling Technology (Beverly, MA, USA). CYR61, HA probe, and YAP1 were purchased from Santa Cruz Biotechnology, Inc. (Santa Cruz, CA, USA). Vinculin was purchased from Sigma-Aldrich (St. Louis, MA, USA). Caspase-3 was purchased from Novus Biologicals (Littleton, CO, USA). MST1 and TEF-1 were obtained from BD Biosciences (San Jose, CA, USA). Horseradish peroxidase-conjugated secondary antibodies were purchased from GE Healthcare (Chicago, IL, USA). Alexa Fluor 488-conjugated goat anti-mouse IgG (H+L) antibody and Alexa Fluor 594-conjugated goat anti-rabbit IgG (H+L) antibody were purchased from Invitrogen (Carlsbad, CA, USA).

### Chemicals

Isoalantolactone (IALT) was purchased from MedChemExpress (HY-N0780; MCE, Monmouth Junction, NJ, USA) and Sigma-Aldrich (470-17-7; Burlington, MA, USA). Phos-tag-conjugated acrylamide was purchased from Wako Chemicals (304-93521; Richmond, VA, USA).

### Cell Culture and Transfection

SNU478, SNU478 LATS1/LATS2 KO, SNU1079, and SNU1196 cells were cultured in RPMI 1640 medium supplemented with 10% fetal bovine serum (FBS), 50 units/mL penicillin, and 50 g/mL streptomycin. HEK293A MAP4K4/6/7 KO, HEK293A MST1/MST2 KO, HEK293A LATS1/LATS2 KO, HEK293A, and HEK293T cells were cultured in Dulbecco's Modified Eagle's Medium (DMEM) supplemented with 10% FBS, 50 units/mL penicillin, and 50 μg/mL streptomycin. All cells were maintained under humidified conditions in a 5% CO 2 incubator. HEK293A, HEK293A MAP4K4/6/7 KO, HEK293A MST1/MST2 KO, HEK293A LATS1/LATS2 KO, and HEK293T cells were kindly provided by Dr. Kun-Liang Guan (University of California, San Diego, USA), and SNU478, SNU1079, and SNU1196 cells were obtained from the Korean Cell Line Bank (KCLB; Seoul, Republic of Korea). Plasmid transfection was performed using polyethylenimine (PEI, Polysciences, Inc., Warrington, PA, USA) at appropriate concentrations of each plasmid DNA according to the manufacturer's instructions.

### CRISPR/Cas9 System

SNU478 LATS1/LATS2 KO cells were generated using the CRISPR/Cas9 system. Single guide RNAs (sgRNAs) were designed using a CRISPR design tool and cloned into the pSpCas9(BB)-2A-Puro and lentiCRISPR V2 vectors provided by Addgene. Lentiviral clones generated from the lentiCRISPR V2 vector were transduced into SNU478 cells. After 24 hours of transduction or infection, SNU478 cells were selected for two days in medium containing puromycin. The cells were then isolated as single-cell clones.

### Retroviral Infection and Establishment of Stable Cell Lines

To generate retrovirus, HEK293T cells were transfected with the pPGS-HA vector or HA-TEAD1ΔC-YAP(AD) construct, together with viral envelope and packaging plasmids, using polyethylenimine. After 48 hours of incubation, the retroviral supernatant was filtered through a 0.45-μm syringe filter and used to infect SNU478 cells with polybrene. The infected cells expressing HA-TEAD1ΔC-YAP(AD) were selected in culture medium containing G418.

### Cell Lysis and Western Blotting

Cells were lysed in lysis buffer containing 40% glycerol, 8% SDS, 20% (3-mercaptoethanol, 0.2 M Tris (pH 6.8), and 0.04% bromophenol blue. The cell lysates were heated at 100° C. and centrifuged at 13,000 rpm for 5 minutes. The supernatants were separated by SDS-PAGE on 8-15% gels and transferred onto PVDF membranes. The membranes were blocked with 1×TBS-T containing 5% skim milk on a shaker and incubated overnight at 4° C. with the primary antibody. The next day, the blots were washed three times and incubated at room temperature with anti-rabbit or anti-mouse HRP-conjugated secondary antibodies diluted 1:5000 in 1×TBS-T containing 5% skim milk. The blots were detected using Immobilon Western Chemiluminescent HRP Substrate. Phos-tag gels were prepared according to the manufacturer's instructions. Band intensities were quantified using ImageJ software.

### MTT Assay

SNU478 WT and SNU478 LATS1/LATS2 KO cells were seeded into 96-well plates. The following day, the cells were treated with various concentrations of IALT and incubated at 37° C. After 2 days, yellow tetrazolium salt (MTT) solution was added to the plates. Absorbance was measured at 570 nm using a microplate spectrophotometer. The IC₅₀ values were calculated from the concentration-response curves using Prism 8.4.3 software.

### Apoptosis Analysis

Apoptosis analysis was performed according to the protocol of the FITC Annexin V Apoptosis Detection Kit. SNU478 WT and LATS1/LATS2 KO cells were seeded into 6-well plates and treated with 0 (DMSO), 5, or 10 M IALT for 48 hours. Adherent cells were detached by trypsinization and collected by centrifugation. The cell pellets were washed with PBS twice and resuspended in 1×binding buffer. Each sample was then stained with 3 μL of propidium iodide (PI) and 3 μL of Annexin V, and early and late apoptosis were analyzed using the BD FACSAria III Cell Sorter at the Three-Dimensional Immune System Imaging Core Facility of Ajou University.

### Immunoprecipitation (IP)

To evaluate protein-protein interactions, cells were lysed in a mild lysis buffer (10 mM Tris, pH 7.5; 1 mM Na₃VO₄; 100 mM NaCl; 1 mM EDTA; 50 mM NaF; 1% NP-40; 2 μg/mL leupeptin; and aprotinin) and centrifuged at 13,000 rpm for 15 minutes at 4° C. The supernatant was incubated with the indicated antibody for 2 hours at 4° C. on a 3600 rotator. Protein A/G magnetic beads were washed with mild lysis buffer and added to the lysates, followed by incubation for 1 hour at 4° C. with rotation. The immunoprecipitates were washed three times with mild lysis buffer and eluted in protein sample buffer.

### Immunocytochemistry

SNU478 WT and LATS1/LATS2 KO cells were seeded on coverslips placed in 6-well culture plates. The cells were fixed with 2% formaldehyde for 15 minutes at room temperature and permeabilized with 0.2% Triton X-100 for 5 minutes. After blocking with 1×PBS containing 10% FBS for 30 minutes, the cells were incubated overnight at 4° C. with the primary antibody in blocking buffer. The cells were washed with 1×PBS containing 10% FBS and incubated for 1 hour and 30 minutes at 25° C. with Alexa Fluor 488-conjugated goat anti-mouse IgG (H+L) and Alexa Fluor 594-conjugated goat anti-rabbit IgG (H+L) secondary antibodies. After washing three times with 1×PBS, the nuclei were counterstained with DAPI for 2 minutes. The coverslips were mounted using Gel/Mount and dried at 4° C. Microscopic observation was performed using an LSM980 NLO multiphoton microscope at the Three-Dimensional Immune System Imaging Core Facility of Ajou University. ZEN (blue edition) software was used for data analysis.

RNA Isolation and qRT-PCR

Total RNA was extracted using TRIzol reagent. Complementary DNA (cDNA) was synthesized from 1 μg of total RNA using reverse transcriptase, 25 mM MgCl₂, 5×reaction buffer, recombinant RNase inhibitor, dNTPs, and random primers. Quantitative real-time PCR (qRT-PCR) was performed using the KAPA SYBR FAST qPCR Master Mix (2×) kit with appropriate primer pairs, according to the manufacturer's instructions, and data were collected using the StepOnePlus Real-Time PCR System. Expression levels were normalized to hypoxanthine-guanine phosphoribosyltransferase 1 (HPRT1). The sequences of the qPCR primers are shown in Table 1 below.

TABLE 1

| Primer | Forward (5′→3′) | Reverse (5′→3′) |
|---|---|---|
| CTGF | CCAATGACAACGCCTC | TGGTGCAGCCAGAAAGCTC |
| | CTG | |
| CYR61 | AGCCTCGCATCCTATA | TTCTTTCACAAGGCGGCACTC |
| | CAACC | |
| HPRT | AGAATGTCTTGATTGT | ACCTTGACCATCTTTGGATTA |
| | GGAAGA | |

### Sulforhodamine B (SRB) Assay

SNU478 WT and SNU478 LATS1/LATS2 KO cells were seeded into 96-well culture plates. The plates were incubated at 37° C., and cells were harvested at the same time every two days. The plates were fixed overnight at 4° C. with 10% trichloroacetic acid solution and washed three times with distilled water. The cells were stained with 0.4% sulforhodamine B in 0.1% acetic acid solution for approximately 30 minutes at room temperature in the dark. The stained cells were then dissolved in 10 mM Trizma base on a shaker for at least 1 hour. The absorbance of the dissolved cells was measured at 540 nm using a microplate spectrophotometer.

### Wound-Healing Assay

SNU478 cells expressing either pPGS-HA (empty vector) or HA-TEAD1ΔC-YAP(AD) were cultured in 12-well plates at 37° C. until approximately 90% confluence was reached. The cell monolayer was scratched using a sterile pipette tip, and the medium was replaced to remove detached cells. The wounded area was monitored for 48 hours using the ZEISS Celldiscover7 microscope at the Three-Dimensional Immune System Imaging Core Facility of Ajou University, and cell migration was analyzed using the ZEN 3.5 (blue edition) software.

### Clonogenic Cell Survival Assay

SNU478 WT and SNU478 LATS1/LATS2 KO cells were seeded into 12-well plates and incubated at 37° C. for approximately two weeks. During incubation, the medium containing IALT was replaced every three days. After about two weeks, the cells were stained with 0.25% crystal violet for 5-10 minutes and washed with distilled water until the background was cleared. The cells were destained in 95% ethanol on a shaker for 1 hour, and the absorbance of the destaining solution was measured at 595 nm using a microplate spectrophotometer.

### TA Cloning

The AccuRapid TA Cloning Kit was used for cloning. First, genomic DNA was extracted from knockout cells using the AccuPrep Genomic DNA Extraction Kit, and PCR was performed using Taq polymerase. The PCR products were purified using the Prep GEL/PCR Purification Mini Kit to obtain insert DNA, which was confirmed by electrophoresis. The insert DNA was then mixed with the pBHA-T vector, T4 ligase, and reaction buffer, followed by incubation at 16° C. overnight. The recombinant plasmid was transformed into competent E. coli DH5a cells, which were spread onto LB agar plates containing ampicillin, IPTG, and X-Gal, and incubated at 37° C. to distinguish colonies. After overnight incubation, white colonies containing the insert were collected from the plates, and colony PCR was performed. The white colonies confirmed by PCR were analyzed through gel electrophoresis, compared with the positive control, and verified using the NCBI Basic Local Alignment Search Tool (BLAST).

### Tumor Isolation in Mice

Five-week-old (18-21 g) BALB/c nude mice were purchased from Orient Bio, Inc. In vitro cultured SNU478 cells were subcutaneously injected into six-week-old male nude mice. Twenty-four days after cell injection, the mice were randomly divided into two groups (n=5 per group). Isoalantolactone (IALT, 10 mg/kg) diluted in less than 5% DMSO and sunflower seed oil was intraperitoneally administered every three days to the IALT-treated group. The control (Con) group received only the vehicle containing less than 5% DMSO and sunflower seed oil. Tumor size was measured every four days. After 24 days of drug administration, the mice were sacrificed, and tumor weights were analyzed. The animal experimental protocols used in this study were reviewed and approved by the Institutional Animal Care and Use Committee (IACUC) of Ajou University Medical Center, ensuring compliance with ethical and scientific management standards.

### Statistical Analysis

Statistical analysis was performed using GraphPad Prism software version 8.4.3. The results are expressed as the mean±standard error of the mean (SEM) from three independent experiments or three independent biological replicates. Statistical significance was determined using an unpaired, two-tailed Student's t-test or two-way ANOVA, where *P<0.05, **P<0.01, ***P<0.001, ****P<0.0001 were considered statistically significant. No statistical methods were used to predetermine sample size. The investigators were blinded to group allocation during the experiment and data analysis.

### [Example 1] Effect of IALT on Cell Viability and Apoptosis Induction in SNU478 Cells

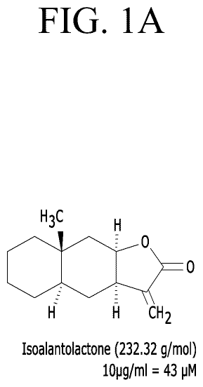

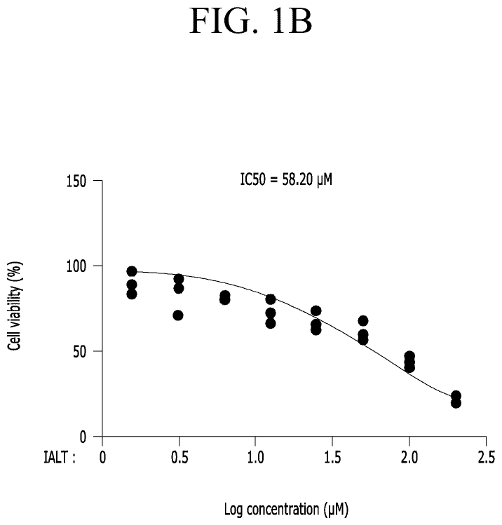

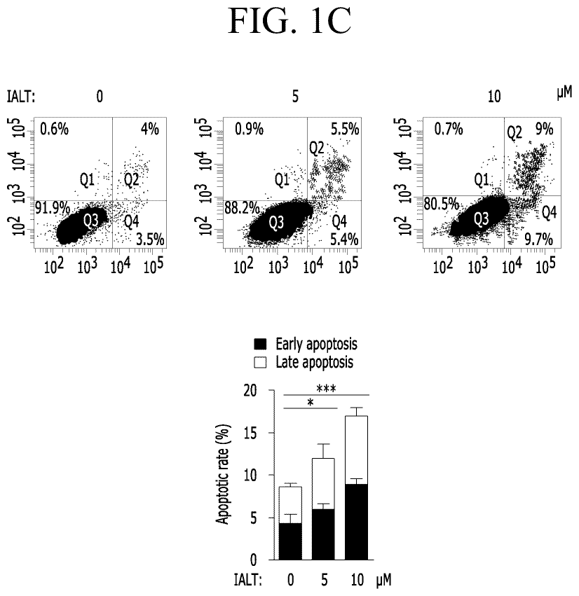

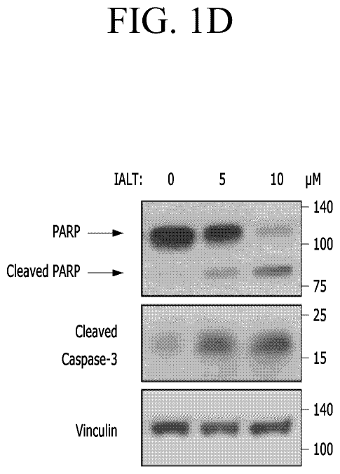

The cytotoxicity of isoalantolactone (IALT), a sesquiterpene lactone derived from medicinal plants such as Inula helenium and Vernonia amygdalina, was evaluated in SNU478 cells (FIG. 1A). Cell viability was assessed using an MTT assay after treating the cells with various concentrations of IALT. Following 48 hours of incubation, the IC₅₀ value of IALT was determined to be 58.2 M (FIG. 1). In addition, the apoptotic potential of IALT was evaluated by Annexin V/PI staining and by examining the expression levels of apoptosis-related proteins such as PARP and cleaved caspase-3 (FIGS. 1C and 1D). In the absence of IALT, SNU478 cells remained viable; however, in the presence of IALT, Annexin V-positive apoptotic cells were induced (FIG. 1C). Furthermore, Western blot analysis was performed to assess caspase activation during apoptosis, and the presence of cleaved caspase-3 was observed in IALT-treated cells. Caspase activation was further confirmed by the cleavage of PARP, a substrate of caspase-3 (FIG. 1D). Notably, IALT exhibited a strong ability to induce apoptosis, as evidenced by the marked cleavage of PARP and caspase-3 at a concentration of 10 M (FIG. 1D). These findings provide strong evidence of the apoptotic effects of IALT in SNU478 cells

### [Example 2] Induction of YAP Phosphorylation and Inhibition of the YAP-TEAD Transcriptional Complex by IALT in Cholangiocarcinoma Cells

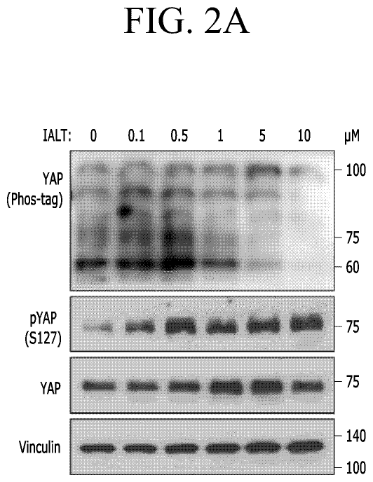

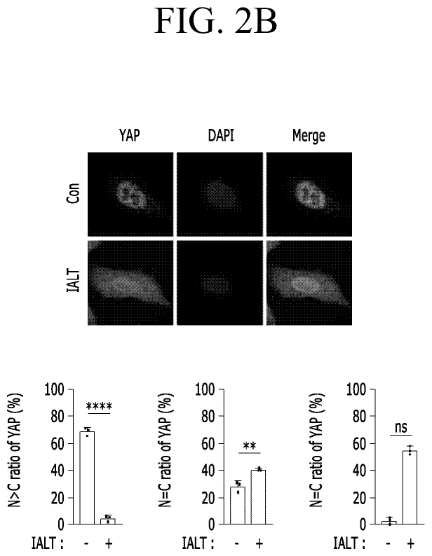

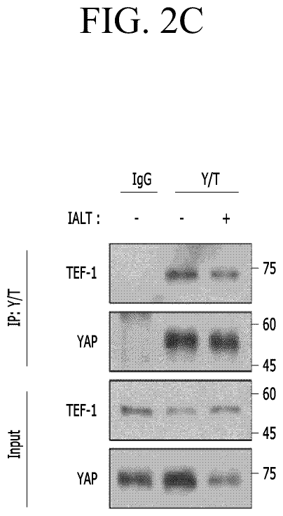

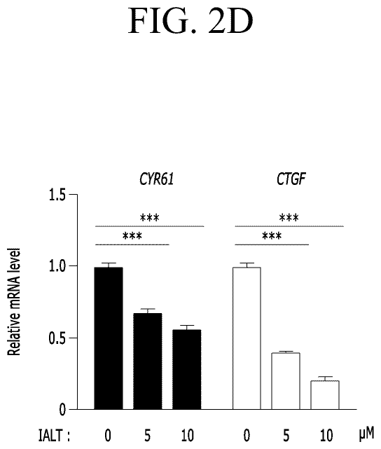

The inhibitory effect of isoalantolactone (IALT) on YAP, known to be activated in cholangiocarcinoma cells such as SNU478, was investigated. The phosphorylation status of YAP was evaluated using Phos-tag gel mobility shift assay and immunoblotting with an anti-pYAP (Ser127) antibody. As a result, IALT treatment induced concentration-dependent phosphorylation of both YAP and TAZ in SNU478 cells (FIG. 2A). Similar effects were observed in other cholangiocarcinoma cell lines, SNU1079 and SNU1196, further confirming the ability of IALT to promote YAP phosphorylation. These findings demonstrate that IALT induces YAP phosphorylation in cholangiocarcinoma cells.

To examine the effect of IALT treatment on the intracellular localization of phosphorylated YAP, immunofluorescence staining was performed. In the absence of IALT, YAP was predominantly localized in the nucleus. However, upon IALT treatment, YAP was markedly translocated from the nucleus to the cytoplasm (FIG. 2B). These results suggest that IALT induces cytoplasmic sequestration of YAP, potentially affecting its function as a transcriptional co-activator. Importantly, the intracellular localization of YAP is closely related to its transcriptional activity, as nuclear YAP promotes gene transcription by forming a complex with the transcription factor TEAD. Consistent with this, IALT treatment decreased the interaction between YAP and TEAD (FIG. 2C). Moreover, IALT treatment reduced the mRNA levels of YAP-dependent genes, including CYR61 and CTGF (FIG. 2D). Collectively, these results demonstrate that IALT suppresses the YAP-TEAD transcriptional complex and consequently affects downstream gene expression. In summary, these findings indicate that IALT induces YAP phosphorylation, disrupts its interaction with TEAD, and regulates the expression of downstream target genes in SNU478 cells.

### [Example 3] Inhibitory Effect of IALT on YAP Activity and Downstream Gene Expression Through Activation of the Canonical Hippo Pathway

The inhibitory effect of isoalantolactone (IALT) on YAP phosphorylation was investigated using knockout (KO) HEK293A cell lines targeting major components of the Hippo signaling pathway, including MST1/MST2, MAP4K4/6/7, and LATS1/LATS2. The effect of IALT on YAP phosphorylation was evaluated using Phos-tag gel mobility shift assay and immunoblotting with specific antibodies against pYAP (Ser127), pMOB (Thr35), and pLATS1 (Thr1079). The results showed that IALT-induced YAP phosphorylation was less effective in MST1/MST2 KO and LATS1/LATS2 KO cells compared with wild-type cells. Notably, IALT still induced YAP phosphorylation in MAP4K4/6/7 KO cells, suggesting that MST1/MST2 primarily mediate LATS1 phosphorylation following IALT treatment.

To assess the functional relevance of IALT-induced YAP phosphorylation in HEK293A MST1/MST2 KO and HEK293A LATS1/LATS2 KO cell lines, mRNA levels of CYR61 and CTGF were analyzed using RT-qPCR. Compared with HEK293A wild-type cells, both HEK293A MST1/MST2 KO and HEK293A LATS1/LATS2 KO cells showed a markedly reduced inhibitory effect of IALT on CYR61 and CTGF expression, further supporting that MST1/MST2 and LATS1/LATS2 are involved in the suppression of YAP-dependent gene expression by IALT.

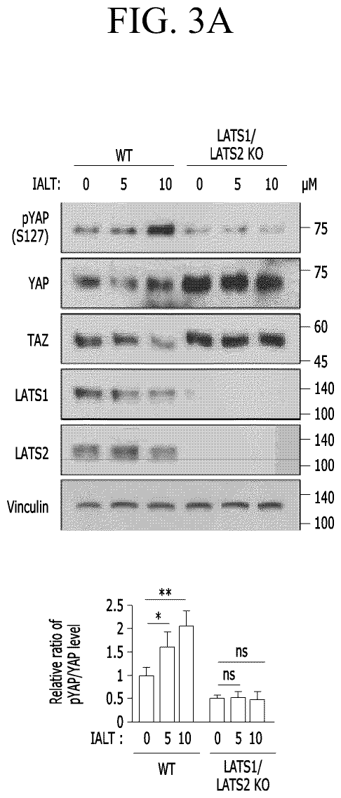

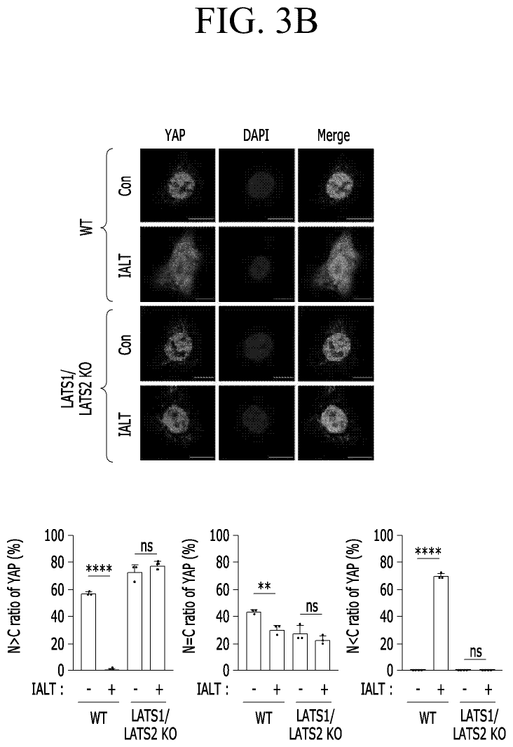

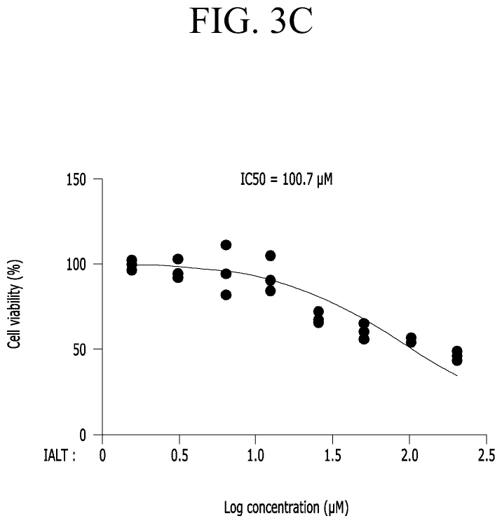

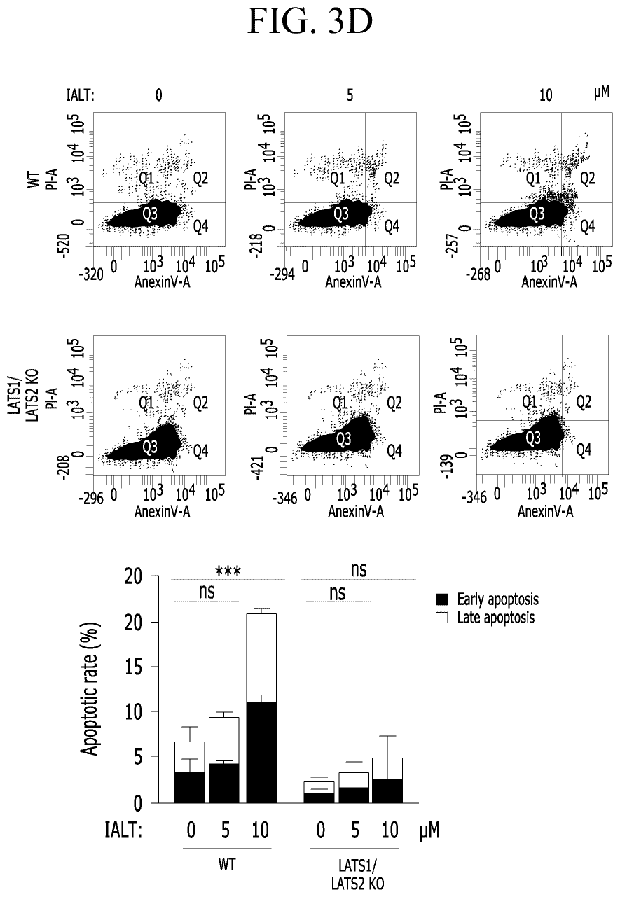

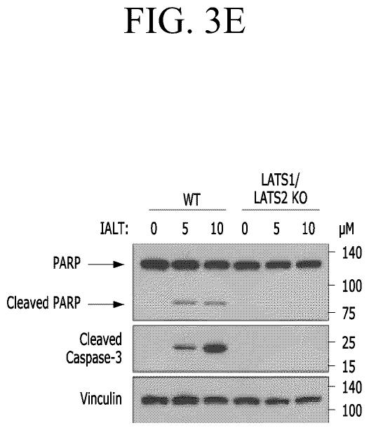

Subsequently, SNU478 cell lines deficient in LATS1/LATS2 were generated using CRISPR/Cas9 technology to evaluate the effect of IALT on LATS1/LATS2-mediated YAP phosphorylation. In these SNU478 LATS1/LATS2 KO cells, IALT failed to induce YAP phosphorylation (FIG. 3A). Moreover, in LATS1/LATS2 KO cells, the ability of IALT to retain YAP in the cytoplasm was lost, resulting in predominant nuclear localization of YAP (FIG. 3B).

Further evaluation of the response of SNU478 LATS1/LATS2 KO cells to various concentrations of IALT revealed that the IC₅₀ value of IALT was 100.7 M (FIG. 3C). Moreover, as observed in MTT assays and morphological assessments, the cytotoxic effect of IALT was reduced in SNU478 LATS1/LATS2 KO cells compared with wild-type cells (FIGS. 1B and 3C). In addition, the apoptotic effect of IALT was attenuated in LATS1/LATS2 knockout cells, as evidenced by PI and Annexin V double staining and by reduced levels of cleaved PARP and cleaved caspase-3 compared with the wild-type cell line (FIGS. 3D and 3E). Overall, these results suggest that IALT suppresses YAP activity through activation of the canonical Hippo pathway, leading to YAP phosphorylation and subsequent regulation of downstream gene expression. These findings provide valuable insights into the molecular mechanism underlying the inhibitory effect of IALT on YAP function.

### [Example 4] Inhibitory Effects of IALT on Cell Growth, Migration, and Tumor Progression Through Suppression of YAP Activity in Cholangiocarcinoma Cells

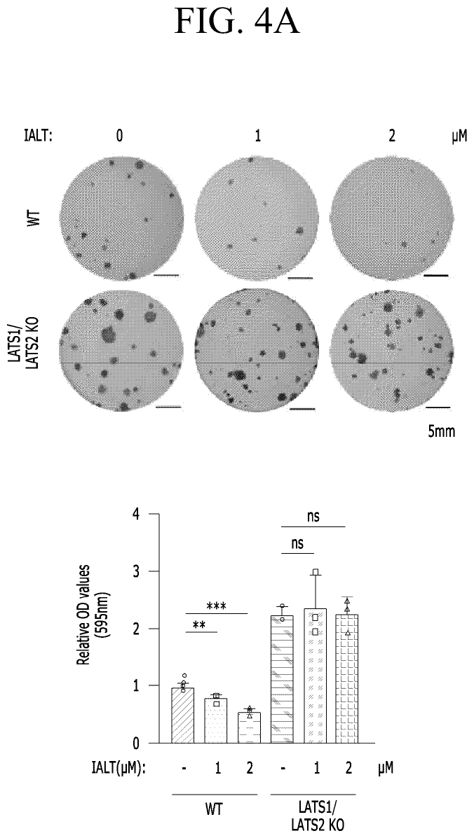

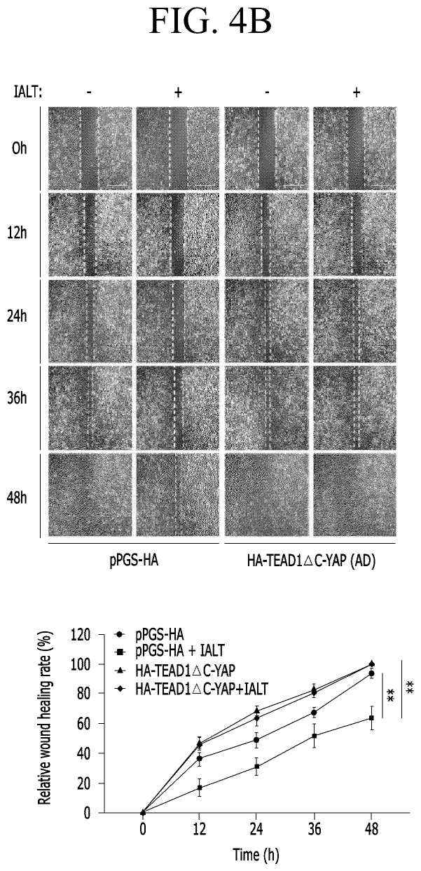

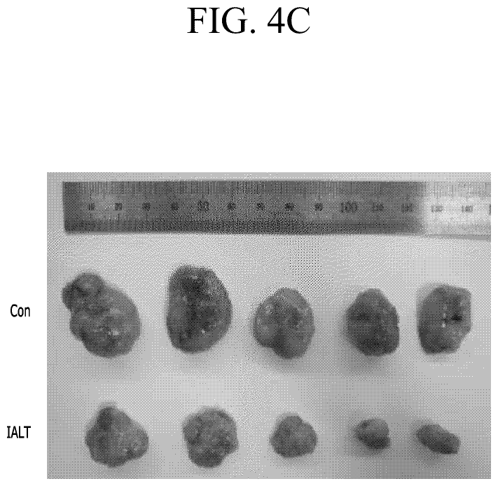

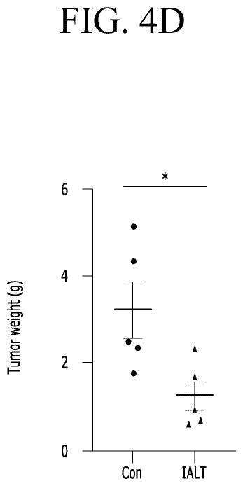

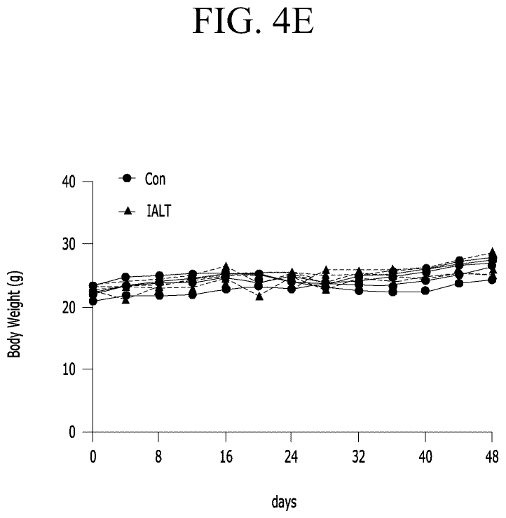

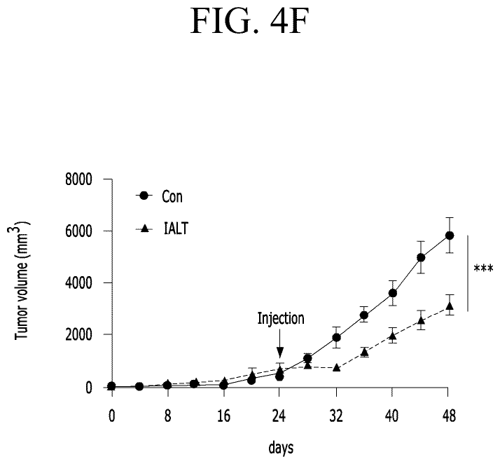

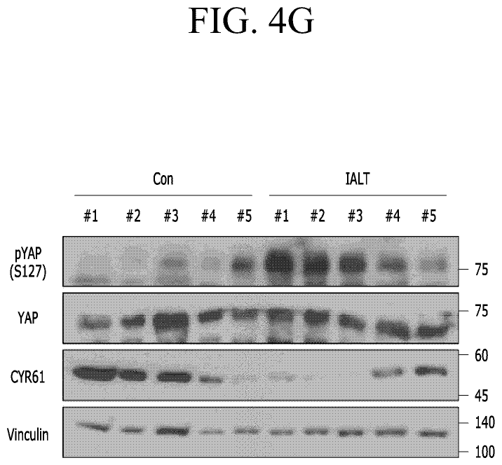

To evaluate the biological effects of isoalantolactone (IALT) on cell growth, a clonogenic growth assay was performed using SNU478 WT and SNU478 LATS1/LATS2 KO cells. Interestingly, the inhibitory effect of IALT on cell growth was attenuated in SNU478 LATS1/LATS2 KO cells (FIG. 4A). In addition, a proliferation assay was conducted to examine the effect of IALT. The results showed that, compared with the control SNU478 WT cells, proliferation was significantly reduced by IALT in WT cells, whereas the deletion of LATS1/LATS2 in SNU478 cells abolished the inhibitory effect of IALT.

To investigate the role of YAP-TEAD activity in IALT-induced inhibition of cell migration, an activated YAP-TEAD fusion protein (TEADΔ1C-YAP(AD)) was introduced into SNU478 cells. Remarkably, the inhibitory effect of IALT on cell migration was lost in SNU478 cells expressing TEAD1ΔC-YAP(AD), directly demonstrating that the suppression of cell migration by IALT is mediated through the inhibition of YAP-TEAD activity (FIG. 4B).

Furthermore, subcutaneous injection of SNU478 cells in an immunodeficient nude mouse xenograft model induced tumor formation (FIG. 4C). Consistent with the in vitro findings, administration of IALT markedly reduced the growth of xenografted tumors in these mice (FIGS. 4D-4G), providing additional evidence of the ability of IALT to suppress YAP activity in vivo. Collectively, these results highlight the ability of IALT to inhibit tumor progression through regulation of YAP activity.

Through the above examples, it was confirmed that IALT exerts biological effects by regulating YAP activity via the canonical Hippo-LATS signaling pathway. By inducing YAP phosphorylation and consequent cytoplasmic sequestration, IALT effectively suppressed YAP-dependent gene expression and inhibited cell growth, migration, and tumor progression. These findings provide valuable insights into the molecular mechanisms underlying the antitumor properties of IALT and emphasize its potential as a promising therapeutic candidate for cancer treatment targeting YAP signaling.

The aforementioned description of the present disclosure is to be exemplified, and it will be understood by those skilled in the art that the present disclosure may be easily modified in other detailed forms without changing the technical spirit or required features of the present disclosure. Therefore, it should be appreciated that the examples described above are illustrative in all aspects and are not restricted. For example, each component described as a singular form may be implemented in a distributed manner, and components described as being distributed may also be implemented in a combined form.

The scope of the present disclosure is represented by appended claims to be described below rather than the detailed description, and it is to be interpreted that the meaning and scope of the claims and all the changes or modified forms derived from the equivalents thereof come within the scope of the present disclosure.

What is claimed is:

1. A composition for modulating the Hippo-YAP pathway, comprising isoalantolactone as an active ingredient.

2. The composition for modulating the Hippo-YAP pathway of claim 1, wherein YAP is phosphorylated by the isoalantolactone.

3. The composition for modulating the Hippo-YAP pathway of claim 1, wherein cytoplasmic sequestration of YAP is induced by the isoalantolactone.

4. The composition for modulating the Hippo-YAP pathway of claim 1, wherein the interaction between YAP and TEAD is inhibited by the isoalantolactone.

5. The composition for modulating the Hippo-YAP pathway of claim 1, wherein the expression of YAP-dependent genes is inhibited by the isoalantolactone.

6. The composition for modulating the Hippo-YAP pathway of claim 5, wherein the YAP-dependent gene comprises at least one selected from the group consisting of CYR61 and CTGF.

7. A pharmaceutical composition for the prevention or treatment of cancer, comprising isoalantolactone as an active ingredient.

8. The pharmaceutical composition for the prevention or treatment of cancer of claim 7, wherein apoptosis of cancer cells is induced by the isoalantolactone.

9. The pharmaceutical composition for the prevention or treatment of cancer of claim 7, wherein tumor growth is inhibited by the isoalantolactone.

10. The pharmaceutical composition for the prevention or treatment of cancer of claim 7, wherein the cancer is selected from the group consisting of cholangiocarcinoma, lung cancer, breast cancer, oral cancer, esophageal cancer, gastric cancer, liver cancer, pancreatic cancer, colorectal cancer, renal cancer, cervical cancer, ovarian cancer, prostate cancer, and combinations thereof.

11. The pharmaceutical composition for the prevention or treatment of cancer of claim 10, wherein the cancer comprises cholangiocarcinoma.

12. The pharmaceutical composition for the prevention or treatment of cancer of claim 7, wherein the pharmaceutical composition further comprises a pharmaceutically acceptable carrier.

13. The pharmaceutical composition for the prevention or treatment of cancer of claim 12, wherein the carrier comprises one selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, menthol, mineral oil, and combinations thereof.

14. The pharmaceutical composition for the prevention or treatment of cancer of claim 12, wherein the pharmaceutical composition further comprises an ingredient selected from the group consisting of preservatives, solubilizers, stabilizers, wetting agents, sweeteners, colorants, flavoring agents, salts, buffers, antioxidants, lubricants, emulsifiers, suspending agents, and combinations thereof.

15. The pharmaceutical composition for the prevention or treatment of cancer of claim 12, wherein the pharmaceutical composition is administered by a method selected from the group consisting of intraperitoneal administration, oral administration, inhalation administration, intravenous administration, intramuscular administration, subcutaneous administration, intradermal administration, intrauterine administration, tumor administration, rectal administration, and combinations thereof.

16. A kit for the prevention or treatment of cancer, comprising the pharmaceutical composition for the prevention or treatment of cancer according to any one of claim 7.
